(19) United States
(12) Patent Application Publication
(10) Pub. No.: US 2023/0302223 A1
(43) Pub. Date: Sep. 28, 2023

(54) A MEDICAMENT DELIVERY DEVICE PACKAGE ASSEMBLY

(71) Applicant: SHL MEDICAL AG, ZUG (CH)

(72) Inventors: Ramazan Gurkan Demirozer, Boca Raton, FL (US); Slobodan Stefanov, Deerfield Beach, FL (US); Ramin Rafiei, Woollahra (AU)

(21) Appl. No.: 18/020,331

(22) PCT Filed: Aug. 12, 2021
(86) PCT No.: PCT/EP2021/072484

§ 371 Date: Feb. 8, 2023

Related U.S. Application Data
(60) Provisional application No. 63/070,337, filed on Aug. 26, 2020.

(30) Foreign Application Priority Data
Oct. 22, 2020 (EP) 20203412.0

(51) Int. Cl. A61M 5/20

(52) U.S. Cl. CPC A61M 5/20; A61M 2205/14; A61M 2205/3368; A61M 2205/50

(57) ABSTRACT

A medicament delivery device package assembly is provided having a body defining an interior reservoir for containing a medicament delivery device; a motion sensor attached to the body; wherein the medicament delivery device package assembly is configured to be placed in a storage unit with a door; wherein the door is movable between a closed position where an inner space of the storage unit is covered by the door and an open position where the inner space of the storage unit is not covered by the door; and wherein the motion sensor is configured to detect an opening movement of the door from the closed position to the open position.

### CROSS REFERENCE TO RELATED APPLICATIONS

The present application is a U.S. National Phase application pursuant to 35 U.S.C. § 371 of International Application No. PCT/EP2021/072484 filed Aug. 12, 2021, which claims priority to U.S. Patent Application No. 63/070,337 filed Aug. 26, 2020 and European Patent Application No. 20203412.0 filed Oct. 22, 2020. The entire disclosure contents of these applications are herewith incorporated by reference into the present application.

### TECHNICAL FIELD

The present disclosure generally relates to a package assembly, and especially to a medicament delivery device package assembly for storing a medicament delivery device.

### BACKGROUND

Portable medicament delivery devices such as auto-injectors, inhalers and on-body devices are generally known for the self-administration of a medicament by patients without formal medical training or for emergency use. For example, patients suffering from diabetes or people undertaking an artificial fertilization procedure may require repeated injections of insulin or a hormone. Other patients may require regular injections of other types of medicaments, such as a growth hormone.

To facilitate such users, there is a demand to have a medicament delivery device with a prefilled medicament container, so that the users can easily and safely carry out the self-administration of a medicament in their home. A medicament may have a predefined dosing schedule which requires the administration of a dose at relatively long intervals, for instance every two or four weeks, or once a month. The medicament may be provided in one single medicament delivery device for multi-use or in multiple medicament delivery devices. The medicament may need to be stored at a certain temperature. The medicament delivery device(s) may be stored in a packaging assembly, which may be stored in the fridge for 1 to 6 months or even longer for instance.

It can be difficult for both the users and the caregivers, e.g. doctors, to keep track of each scheduled dosing time. There is therefore a demand for providing a medicament delivery device package assembly with an indication, sensors and a communication unit, so that the users can get a notification, by the indication, about the previous/next dosing time, and the caregiver can also get a notification from the communication unit about the users' intake of the medicament.

However, package assemblies with an indication, sensors and a communication unit, especially a communication unit based on cellular wireless communication technology, usually require high power consumption. It has been appreciated that there is a need for improved designs to help regulate the power consumption of the medicament delivery device package assembly. Also, for emergency use medicament delivery devices, there is a demand to provide a significant indication to indicate the location of such emergency use medicament delivery devices especially if they are stored with other items.

### SUMMARY

The present disclosure is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially”, “rotation”, “rotational” and “rotationally” refer to a direction generally perpendicular to the longitudinal direction and at least partially extending around the longitudinal direction.

There is hence provided a medicament delivery device package assembly comprising: a body defining an interior reservoir for containing a medicament delivery device; a motion sensor attached to the body; wherein the medicament delivery device package assembly is configured to be placed in a storage unit with a door, wherein the door is movable between a closed position where an inner space of the storage unit is covered by the door and an open position where the inner space of the storage unit is not covered by the door; and wherein the motion sensor is configured to detect an opening movement of the door from the closed position to the open position.

The opening movement of the door of storage can be deemed as one of signs of accessing the medicament delivery device package assembly, so the detection of the opening movement of the door can be used to regulate the power consumption of the medicament delivery device package assembly, or triggering the events that may be related to use of the contained medicament delivery devices, such as providing a user guidance on the body or triggering a data transmission.

According to one embodiment, the motion sensor is configured to detect a relative movement between the door and the body.

According to one embodiment, the motion sensor is a proximity sensor; the motion sensor is configured to detect a change of a distance between the door and the body.

According to one embodiment, the motion sensor is configured to detect an increase of a distance between the door and the body.

According to one embodiment, the motion sensor is configured to detect a presence or an absence of the door in a detectable range of the motion sensor.

According to one embodiment, the motion sensor is an inertia sensor; the motion sensor is configured to detect the movement of the body together with the door.

According to one embodiment, the medicament delivery device package assembly comprises an indicator arranged on the body; when the motion sensor detects an opening movement of the door from the closed position to the open position, the motion sensor is configured to trigger the indicator arranged on the body of the package assembly to provide an indication to a user.

According to one embodiment, the medicament delivery device package assembly comprises a set of electronics; when the motion sensor detects an opening movement of the door from the closed position to the open position, the motion sensor is configured to wake up the set of electronics from a sleep mode to a working mode.

According to one embodiment, the medicament delivery device package assembly comprises a communication unit; when the motion sensor detects an opening movement of the door from the closed position to the open position, the motion sensor is configured to trigger the communication unit to send out a signal.

According to one embodiment, the medicament delivery device package assembly comprises a reservoir sensor; the reservoir sensor is arranged within the interior reservoir; and the reservoir sensor is configured to detect a removal of a medicament delivery device from the interior reservoir.

According to one embodiment, the medicament delivery device package assembly is configured to store multiple medicament delivery devices.

According to one embodiment, the interior reservoir of the medicament delivery device package assembly is divided into multiple chambers for respectively containing a medicament delivery device.

According to one embodiment, the reservoir sensor is arranged within each one of chambers.

According to one embodiment, the medicament delivery device package assembly comprises an opening, with the opening connected to the interior reservoir; the medicament delivery device package assembly comprises an opening sensor configured to detect whether the opening is covered.

According to one embodiment, the medicament delivery device package assembly comprises a movable lid arranged on the body; the lid is movable between a lid closed position, in which the opening is covered by the lid; and an lid open position, in which the opening is not covered by the lid; and the opening sensor is configured to detect a contact between the lid and the body or a movement of the lid from the closed position to the open position.

According to one embodiment, the movable lid comprises a safety lock, e.g. a mechanical lock, an electromagnetic lock, a magnetic lock, and/or an electrometrical lock.

According to one embodiment, the safety lock can be locked and unlocked by a key mechanism using mechanical way, electronic way, and/or biological identification, such as a movement of a matching mechanical key, inputting of a set of code, fingerprint, and/or facial identifying.

According to one embodiment, the lid is connected with the body by a hinge.

According to one embodiment, when the medicament delivery device package assembly comprises the reservoir sensor, the reservoir sensor can be one of an electromechanical switch, a light sensor, a proximity sensor, a capacitive sensor, and an infra-red sensor; and when the medicament delivery device package assembly comprises the opening sensor, the opening sensor can be one of an electromechanical switch, a light sensor, a proximity sensor, a capacitive sensor, and an infra-red sensor.

According to one embodiment, the medicament delivery device package assembly comprise a temperature sensor.

According to one embodiment, the temperature sensor being configured to detect an ambient temperature, a storage temperature within the medicament delivery device package assembly, and/or a temperature of the medicament contained within the medicament delivery device that is stored within the medicament delivery device package assembly. The temperature sensor may be always in an activated state when the general switch has been switched on.

According to another aspect of the present disclosure provided a method of operating a medicament delivery device package assembly configured to be placed in a storage unit with a door, the medicament delivery device package assembly comprising a body defining an interior reservoir for containing a medicament delivery device and a motion sensor attached to the body, the method comprising the steps of: detecting the movement of said door from a closed position to an open position with the motion sensor and carrying out one or more of the following steps in response to detecting the movement of said door from the closed position to the open position: triggering an indicator arranged on the body of the package assembly to provide an indication to a user; waking up a set of electronics of the package assembly from a sleep mode to a working mode upon detecting the movement of the door from the closed position to the open position; and triggering a communication unit of the package assembly to send out a signal.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the present disclosure will now be described, by way of example only, with reference to the accompanying drawings, in which:

FIG. 1A-1B schematically show a perspective view of a medicament delivery device package assembly containing a medicament delivery device.

FIG. 2A schematically shows one embodiment of the medicament delivery device package assembly of FIG. 1A-1B being placed in a refrigerator.

FIG. 2B schematically shows another embodiment of the medicament delivery device package assembly of FIG. 1A-1B being placed in a refrigerator.

FIG. 3 schematically shows a perspective view of another embodiment of a medicament delivery device package assembly containing a medicament delivery device.

FIG. 4 schematically shows an electronic arrangement of the medicament delivery device package assembly of FIG. 1A-1B or FIG. 3 in one embodiment.

FIG. 5 shows a flow chart of an operation of the electronic arrangements of FIG. 4 in one embodiment.

### DETAILED DESCRIPTION

Embodiments of the present disclosure provide a medicament delivery device package assembly for storing one or more prefilled medicament delivery devices, e.g. injectors, inhalers or medical sprayers.

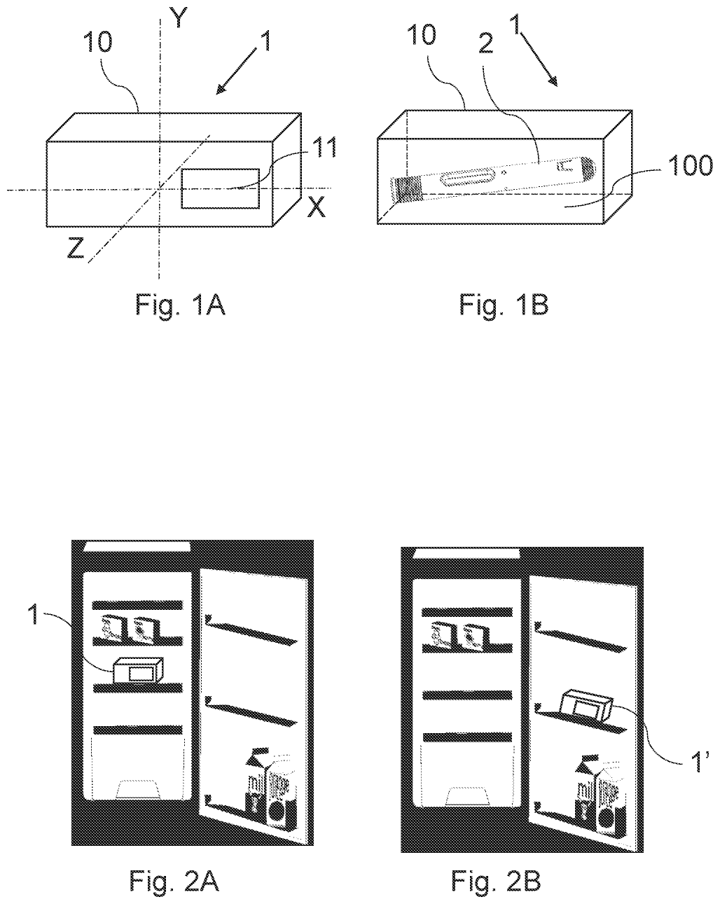

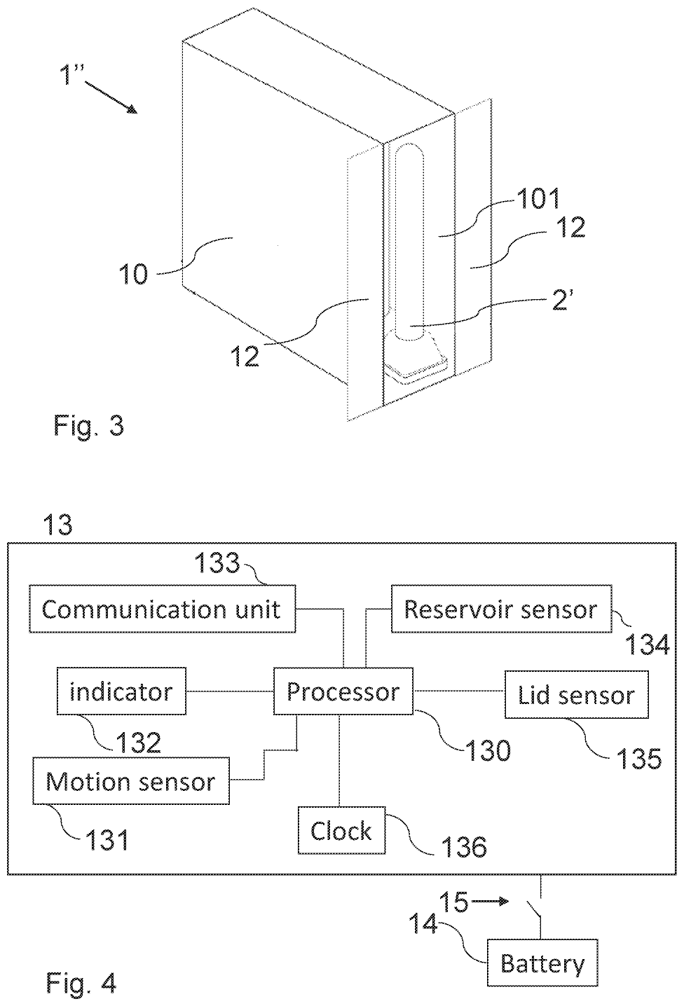

The medicament delivery device package assembly comprises a body defining an interior reservoir so that one or more medicament delivery devices can be contained therein. The body extends along an X axis, a Y axis, and a Z axis that is perpendicular to both the X and Y axes, as shown in FIG. 1A. The medicament delivery device package assembly comprises a motion sensor attached to the body; the motion sensor configured to detect a movement of an object adjacent to the body 10 (this will be explained in detail later). The object is not part of the medicament delivery device package assembly and can move independently relative to the medicament delivery device package assembly. As shown in FIG. 1A-1B and FIG. 3; the exemplified medicament delivery device package assembly 1; 1′; 1″ comprises the body 10 defining the interior reservoir 100 so that one or more medicament delivery devices 2; 2′ can be contained therein.

Besides the motion sensor, the medicament delivery device package assembly 1 comprises a processor unit and may comprise a set of electronics attached to the body. The set of electronics may comprise one or different type of indicators; one or different type of communication units; different types of sensors, e.g. a reservoir sensor, an opening sensor of the medicament delivery device package assembly (will be explained in detail later); or any combination thereof. In a preferred embodiment, the medicament delivery device package assembly comprises an indicator and a communication unit attached to the body.

The processor, the motion sensor and the set of electronics of the medicament delivery device package assembly are preferably powered by a battery, e.g. single use or rechargeable. The battery and the processor, the motion sensor and the set of electronics of the medicament delivery device package assembly can be placed on a printed circuit board (PCB) attached to the body; or the battery may be independently attached to the body and connected to the PCB (containing the processor, the motion sensor and the set of electronics) by wires. The battery may be connected with a general switch, so that the user can switch on/off such general switch to turn on/off a power supply from the battery.

The indicator is configured to provide an indication to the user, e.g. a visual, audible or tactile indication. For example, the indicator can be a LED/LCD screen 11, as shown in FIG. 1A, to provide at least one of a dosing reminder, a usage instruction and a dosing history record to the user. The indicator can also be a touch screen so that it can provide information to the user and also obtain a user input. The indicator is configured to provide an indication for the user of an information relative to the medicament delivery device package assembly or the medicament delivery device(s) within the medicament delivery device package assembly.

The communication unit is preferably a wireless communication unit, e.g. cellular, GPRS, LTE, 3G, 4G, 5G, WiFi, LPWAN, Bluetooth, Zigbee, RFID, or NFC. In a preferred embodiment, the communication unit is based on LTE-M and/or NB-IoT as the communication technology, so that the medicament delivery device package assembly can be more power efficient. The communication unit is configured to send/receive the information related to the medicament delivery device package assembly to/from a remote server or a user device. Such information can be a time of dosing, the dosing history and/or the storage temperature of the medicament, and/or the expiration date of the medicament, for example, depending on the design selection.

The medicament delivery device package assembly may comprise a reservoir sensor arranged within the interior reservoir; the reservoir sensor configured to detect a removal of a medicament delivery device within the interior reservoir. In one example, the medicament delivery device package assembly is configured to store multiple medicament delivery devices. The interior reservoir can be divided into multiple chambers, so that each medicament delivery device can be placed in a chamber. The reservoir sensor can be attached to each chamber (for example, bottom of an inner surface of the body or around an opening of each chamber). The reservoir sensor can be an electrical circuit with multiple sub-circuits respectively arranged within each chamber, with each sub-circuit comprising a mechanical switch. Each mechanical switch is designed to interact with a medicament delivery device that is placed within the respective chamber, for example, when the medicament delivery device is placed in the chamber, the medicament delivery device may press on the switch, so that a sub-circuit loop is closed, and when such medicament delivery device is removed, the switch may open, so that the sub-circuit loop is open; so that such removal can therefore be detected. Such detection of the removal of the medicament delivery device(s) in the interior reservoir of the body can be used to keep track of the number of the medicament delivery devices in the medicament delivery device package assembly, and to update corresponding data in the remote server or a local smart device.

The medicament delivery device package assembly may also be designed for containing both unused and used medicament delivery devices. In such a case, the interior reservoir can be at least divided into two chambers for respectively containing the unused and used medicament delivery devices; or each chamber can be arranged with another sensor to detect whether a contained medicament delivery device is used or not (especially if the medicament delivery package assembly contains disposable medicament delivery devices); for example, such sensor can be a light sensor to detect the light reflection of a medicament container of the medicament delivery device to determine whether the reflection is by a medicament or other rubber/plastic components; or a mass sensor, so that the usage detection can be done by identifying the difference of a weight between a used and unused medicament delivery devices. The light sensor or the mass sensor can also detect the used volume of the contained medicament, especially if the medicament delivery package assembly contains reusable medicament delivery devices. The medicament delivery device package assembly may further comprise an information reader, e.g. RFID/NFC reader or optical code reader, arranged within the interior reservoir, so that if the contained medicament delivery device(s) is arranged with an information tag, such contained information can be loaded to the medicament delivery device package assembly via the information reader. If the medicament delivery device package assembly contains a used medicament delivery device, the information reader can also obtain information about the medicament delivery operation from the used medicament delivery device, particularly if the medicament delivery device is arranged with a recording unit.

FIG. 3 shows an example in which the body 10 of the medicament delivery device package assembly 1″ comprises an opening 101, with the opening 101 connected to the interior reservoir. The user is able to access and take out the stored medicament delivery device(s) via the opening 101. The medicament delivery device package assembly 1″ may comprise a movable lid 12 arranged on the body 10, e.g. by a hinge. The lid 12 is movable between a lid closed position, in which the opening is covered by the lid, so that the user is blocked from accessing the interior reservoir through the opening; and a lid open position, as shown in FIG. 3, in which the opening is not covered by the lid, so that the user is able to access the interior reservoir through the opening. The medicament delivery device package assembly 1′ may comprise an opening sensor; and the opening sensor is configured to detect whether the opening of the medicament delivery device package assembly 1′ is covered. Such detection of the opening sensor can be done by detecting the movement of the movable lid from the lid closed position to the lid open position; or detecting a contact between the lid and the body; or detecting the rotation of the hinge of the lid. The medicament delivery device package assembly may comprise a safety lock for locking the lid with the body. The safety lock can be electromagnetic, for example. In one example, the movable lid comprises a safety lock, e.g. a mechanical lock, an electromagnetic lock, a magnetic lock, and/or an electrometrical lock. The safety lock can be locked and unlocked by a key mechanism using mechanical way, electronic way, and/or biological identification, such as a movement of a matching mechanical key, inputting of a set of code, fingerprint, and/or facial identifying.

On the other hand, instead of the lid, the opening of the medicament delivery device package assembly can be sealed/unsealed by, for example, a zip; especially if the medicament delivery device package assembly is made of a non-rigid material, e.g. a bag. In this example, the opening sensor is configured to detect whether the opening of the medicament delivery device package assembly is sealed or not, by detecting whether the opening is covered by the zip.

It should be noted that the opening sensor may be programmed to detect a complete opening of the medicament delivery device package assembly, meaning that the opening of the medicament delivery device package assembly is completely exposed to the user; or the opening sensor may be programmed to detect a partial opening of the medicament delivery device package assembly, namely, the opening of the medicament delivery device package assembly is partially exposed to the user and partially covered by the lid or the zip. The former example may be suitable for a medicament delivery device package assembly that contains a user-assembled medicament delivery device, such as a medicament delivery device to which the user needs to attach a needle or a medicament container. The latter example may be suitable for a medicament delivery device package assembly that contains a medicament delivery device with a toxic medicament.

The reservoir sensor and/or the opening sensor can be one of an electromechanical switch, a light sensor, a proximity sensor, a capacitive sensor, and an infra-red sensor.

In a preferred embodiment, the medicament delivery device package assembly is configured to be placed in a storage unit with a door; such door is movable between a closed position where an inner space of the storage is covered by the door, so that a user is prevented from accessing to the inner space of the storage unit; and an open position where the inner space of the storage unit is not covered by the door, so that the user is allowed to access to the inner space of the storage unit. In a preferred embodiment, the closed position of the door is when the door completely covers the inner space of the storage unit, and the open position of the door is when the door is partially or completely moved from covering the inner space of the storage unit.

An example of use of the medicament delivery device package assembly may comprise the following stages:
the medicament delivery device package assembly is loaded with the medicament delivery device(s) by the user or the caregiver;
the user/caregiver switches on the general switch; and
the user/caregiver places the medicament delivery device package assembly in a refrigerator, e.g. household refrigerator (the refrigerator being the storage unit with a door in this example), together with the contained prefilled medicament delivery device(s).

In the preferred embodiment, the motion sensor on the medicament delivery device package assembly is configured to detect the opening movement of the door. The opening movement of the door is defined by the movement of the door from the closed position to the open position. In one example, the motion sensor is configured to detect the opening movement of the refrigerator door (the refrigerator door being the door in this example).

The motion sensor can be a proximity sensor, e.g. can be based on infra-red, ultrasonic, or radar. In this case, the motion sensor is able to detect the relative movement between the body of the medicament delivery device package assembly and the object adjacent to the body. In a preferred embodiment, the object is the door of the storage unit.

As shown in FIG. 2A, the medicament delivery device package assembly 1 should be placed on a stationary shelf without anything positioned between the body of the medicament delivery device package assembly and the refrigerator door; so that when the refrigerator door is moved from a door closed position to a door open position, for example, pivoting from the door closed position to the door open position (the door moves partially along the Z axis in FIG. 1A), the motion sensor is able to detect a change of a distance between the refrigerator door and the body of the medicament delivery device package assembly 1, especially the change of the distance along the Z axis. The change of distance between the refrigerator door and the body can be divided into two scenarios: a) a change of distance when the refrigerator door is moved away from the body, such detection should be an increase of the distance between the refrigerator door and the body; b) a change of distance when the refrigerator door is moved close to the body, such detection should be a decrease of the distance between the refrigerator door and the body. In this example, the processor of the medicament delivery device package assembly may interpret the first scenario, namely that the refrigerator door is moved away from the body (distance increasing), as a detection of the opening movement of the refrigerator door; and ignore the second scenario (because it can be a closing of the refrigerator door or a moving hand). Also, the motion sensor in this embodiment can be used when the medicament delivery device package assembly is placed in a partition with an independent door (for example, a hinged door), the partition arranged inside the refrigerator. In this case, the partition is the storage unit with the door, so that only the opening movement of the door of such partition (the door of the storage unit in this example) will be detected by the motion sensor.

On the other hand, if the door of the storage unit is configured to be slid, in parallel with X axis or Y axis, from the closed position to the open position, the motion sensor is then configured to detect a presence or absence of movement of the door of the storage unit in the detectable range of the motion sensor. For example, the motion sensor can be multiple proximity sensor, so that a continuous movement of the door of the storage can be detected; or one proximity sensor, so that the motion sensor can detect a signal change if the door of the storage is moved from a position that is in front of the body of the medicament delivery device package assembly, to a position that is out of the detectable range of the motion sensor. Also, instead of a proximity sensor, the motion sensor in this embodiment can be a light navigator (such as the sensor used in a mouse of a computer, using a light source to emit a light beam on an adjacent object; and a light sensor to catch the reflection from the object), so a sliding movement of an object (which is the door of the storage unit in this example) adjacent to the body of the medicament delivery device package assembly can be detect by the motion sensor.

Furthermore, the motion sensor can also be an inertia sensor, e.g. an accelerometer or gyroscope. In this case, the motion sensor is able to detect the movement the object adjacent to the body together with the body of the medicament delivery device package assembly. In the preferred embodiment, the object is the door of the storage unit. The medicament delivery device package assembly can be placed in a part that is movable with the door or a drawer. For example, as shown in FIG. 2B, the medicament delivery device package assembly 1′ is placed on a shelf of the refrigerator (the refrigerator is the storage unit with the door in this case) that is arranged on an inner surface of the refrigerator door (as the door in this case); so that when the refrigerator door is moved from the door closed position to the door open position, the medicament delivery device package assembly 1′ will be moved together with the refrigerator door. The processor is configured to interpret such movement, detected by the inertia sensor, as a detection of the opening movement of the refrigerator door. In another example, such medicament delivery device package assembly can also be placed in a partition of the refrigerator such as a drawer, or in another kind of storage unit that is a drawer; so that when the user pulls out the drawer, the medicament delivery device package assembly will move together with the drawer, and the processor will then interpret such movement, detecting by the inertia sensor, as a detection of the opening movement of the door (the drawer itself being the door and the storage unit in this example). This embodiment may provide a better detection, even when another object is positioned between the motion sensor and the door of the storage unit.

The motion sensor can be a combination of at least one proximity sensor and at least one inertia sensor, so that the medicament delivery device package assembly will be more compatible for being placed on a stationary shelf inside the storage or on a movable part (such as a drawer or a shelf on the door) of the storage unit.

When the motion sensor detects an opening movement of the door from the closed position to the open position, the motion sensor is configured to subsequent events depending on the design selection. In one embodiment, the motion detection can act as a trigger of an audible and/or a visual indication. This embodiment may be suitable for a medicament delivery device package assembly that stores emergency medicament delivery devices, such as medicament delivery devices for hypoglycemia. The audible indication and/or visual indication is configured to indicate the location of the package assembly, so even when a user, no matter whether it is a user with training or not, is in a rush, the user can immediately find the medicament delivery device package assembly from the storage unit and therefore get such medicament delivery devices.

In another embodiment, when the motion sensor detects an opening movement of the door from the closed position to the open position, the motion sensor is configured to trigger the communication unit to send out a signal to a remote server or a local personal smart device. The material of the storage unit may block or interfere with the data transmission by the communication unit; for example, refrigerators are commonly made with metal/alloy, so when the medicament delivery device package assembly is placed inside a refrigerator, a wireless data transmission may be interfered by those metal/alloy when the refrigerator door is closed. A better moment for the communication unit to wirelessly send out a signal is the door of the storage unit is in the open position. In this case, the communication unit will be triggered to transmit data when the opening movement of the door is detected by the motion sensor.

In another embodiment, the set of electronics has a sleep mode where the set of electronics only has a minimum power consumption or without power consumption (completely shut off); and a working mode where the set of electronics can work normally with a normal power consumption. The detection from the motion sensor is configured to wake up the set of electronics from a sleep mode to a working mode; such that the detection acts as a wake-up event for other electronics (except the processor and the motion sensor), e.g. the indicator, the communication units and other sensors (if any); such that when the opening movement of the door of the storage unit has been detected, the other electronics will be activated (to the working mode from the sleep mode).

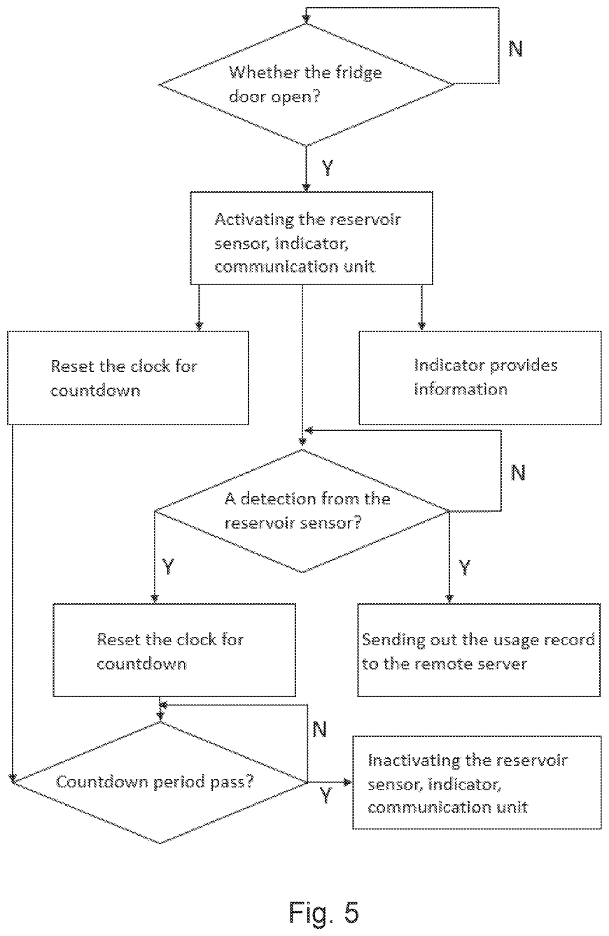

In another embodiment, for regulating the power consumption, the medicament delivery device package assembly may comprise a clock that will be activated when the opening movement of the door of the storage unit has been detected by the motion sensor. The clock is configured to count down a programmed time period and connect with an inactivation mechanism of the set of electronics; so that when such time period passed, the set of electronics is inactivated. In the example shown in FIG. 4-5, the electronics 13 of the medicament delivery device package assembly is connectable with the battery 14; the connection is controlled by the power control switch 15. The electronics 13 comprises the processor 130, the motion sensor 131, the indicator 132, and the communication unit 133. The motion sensor 131, the indicator 132, and the communication unit 133 are connected to the processor 130. In this example, the package assembly 1″ comprises the reservoir sensor 134, the movable lid and the lid sensor 135 (the lid sensor being the opening sensor in this example); the reservoir sensor 134 and the lid sensor 135 are also connected with the processor 130. The processor is also connected to the clock 136.

An example control sequence is shown in FIG. 5. In this embodiment, the refrigerator is the storage unit and the refrigerator door is the door of the storage unit. The indicator 132, the communication unit 133 and the reservoir and the lid sensor 134, 135 will be inactivated after a time period (counted down by the clock), e.g. 30 seconds, if there is no any other event has been detected, e.g. detection of a removal of the medicament delivery device from the interior reservoir or detection of a movement of the movable lid of the medicament delivery device package assembly.

The medicament delivery device package assembly may also comprise a face sensor/eye-contact sensor, e.g. by an image capture unit/camera with image recognition function. In this embodiment, the face sensor/eye-contact sensor may be also arranged as a part of the set of electronics, so it will also be activated only if the opening movement of the door of the storage unit has been detected by the motion sensor. The detection of a face and/or an eye-contact may be configured to control the inactivation mechanism of the set of electronics, e.g. the indicator, the communication unit, or the other sensors (except the motion sensor). For example, the detection of the opening movement of the door of the storage unit is configured to activate the set of electronics. Then if there is subsequently no face and/or eye-contact detected after a short period of time (counted down by the clock), e.g. 15 seconds, the set of electronics will be inactivated (back to the sleep mode from the working mode). Also, the face sensor/eye-contact sensor may be used together with the motion sensor, especially when the motion sensor is a proximity sensor; such that when a face/eye-contact and a change in distance between the body and an object which is moving close to the body are both detected, the processor may interpret such detection as an access intention, so that an indicator may provide a feedback in accordance with such detection. For example, when the access intention has been detected, but there is an medicament taken record (stored in a memory of the medicament delivery device package assembly or downloaded by the communication unit) today, the indicator may provide an alarm to the user. On the other hand, if a change in distance between the body and an object (when the object is moving close to the body) is detected, but there is no detection of a face/eye contact, the processor may then interpret such detection as a closing of the refrigerator door and turn off the indicator, the communication unit and other sensors (if any).

In another embodiment, the medicament delivery device package assembly can also be placed in a storage unit such as: a portable cool insulation box or a portable refrigerator, if the user is going to carry the medicament delivery device and travel. The motion sensor is configured to detect the opening movement of a cover of the portable cool insulation box/portable refrigerator as mentioned above.

The medicament delivery device package assembly may comprise a temperature sensor, the temperature sensor being configured to detect an ambient temperature. The temperature sensor may be always in an activated state when the general switch has been switched on.

In another embodiment, if the medicament delivery device package assembly doesn't need to be stored under certain temperature, e.g. the prefilled medicament only needs to be stored under room temperature, the motion sensor may be configured to detect an opening movement of a door of the storage unit (the storage unit may be a cupboard/sideboard/drawer or a portable carrying case, e.g. medical carrying case), by the mechanism as mentioned above; or the reaching of the user. In the case of detecting the reaching of the user, a change of distance between the body and an object which is moving away from the body, may be ignored by the processor. For example, the proximity sensor is able to detect the reach of the user's hand; the inertia sensor is able to detect the movement of the medicament delivery device package assembly together with the movement of the user's hand when the medicament delivery device package assembly is taken by the user. For better detection, the motion sensor in this embodiment can be arranged together with the face/eye contact sensor, to identify the reaching of the user or a reaching by another object. In this embodiment, the detection from the motion sensor is configured to act as the indication trigger or the wake-up event as mentioned above or to trigger the indicator to provide further information to the user. For example, the motion sensor may trigger the touch screen (as the indicator) to display icons of numbers, so that the user is able to enter a code for unlocking the safety lock of the movable lid. In this embodiment, the body of the medicament delivery device package assembly may be arranged with a cool insulation layer to avoid a significant change of the temperature of the prefilled medicament.

The present concept has mainly been described above with reference to a few examples. However, as is readily appreciated by a person skilled in the art, other embodiments than the ones disclosed above are equally possible within the scope of the present concept, as defined by the appended claims.

1-15. (canceled)

16. A medicament delivery device package assembly comprising:
a body defining an interior reservoir for containing a medicament delivery device;
a motion sensor attached to the body;
wherein the medicament delivery device package assembly is configured to be placed in a storage unit with a door, wherein the door is movable between a closed position where an inner space of the storage unit is covered by the door and an open position where the inner space of the storage unit is not covered by the door; and
wherein the motion sensor is configured to detect an opening movement of the door from the closed position to the open position.

17. The medicament delivery device package assembly as claimed in claim 16, wherein the motion sensor is a proximity sensor; and wherein the motion sensor is configured to detect a change of a distance between the door and the body.

18. The medicament delivery device package assembly as claimed in claim 17, wherein the motion sensor is configured to detect an increase of a distance between the door and the body.

19. The medicament delivery device package assembly as claimed in claim 16, wherein the motion sensor is configured to detect a presence or an absence of movement of the door in a detectable range of the motion sensor.

20. The medicament delivery device package assembly as claimed in claim 16, wherein the motion sensor is an inertia sensor; and wherein the motion sensor is configured to detect the movement of the body together with the door.

21. The medicament delivery device package assembly as claimed in claim 16, wherein the medicament delivery device package assembly comprises an indicator arranged on the body; and wherein when the motion sensor detects an opening movement of the door from the closed position to the open position, the motion sensor is configured to trigger the indicator arranged on the body of the package assembly to provide an indication to a user.

22. The medicament delivery device package assembly as claimed in claim 16, wherein the medicament delivery device package assembly comprises a set of electronics; and wherein when the motion sensor detects an opening movement of the door from the closed position to the open position, the motion sensor is configured to wake up the set of electronics from a sleep mode to a working mode.

23. The medicament delivery device package assembly as claimed in claim 16, wherein the medicament delivery device package assembly comprises a communication unit; and wherein when the motion sensor detects an opening movement of the door from the closed position to the open position, the motion sensor is configured to trigger the communication unit to send out a signal.

24. The medicament delivery device package assembly as claimed in claim 16, wherein the medicament delivery device package assembly comprises a reservoir sensor; wherein the reservoir sensor is arranged within the interior reservoir; and wherein the reservoir sensor is configured to detect a removal of a medicament delivery device from the interior reservoir.

25. The medicament delivery device package assembly as claimed in claim 24, wherein when the medicament delivery device package assembly comprises the reservoir sensor, the reservoir sensor is one of an electromechanical switch, a light sensor, a proximity sensor, a capacitive sensor, and an infra-red sensor.

26. The medicament delivery device package assembly as claimed in claim 16, wherein the medicament delivery device package assembly comprises an opening, with the opening connected to the interior reservoir; and wherein the medicament delivery device package assembly comprises an opening sensor configured to detect whether the opening is covered.

27. The medicament delivery device package assembly as claimed in claim 26, wherein when the medicament delivery device package assembly comprises the opening sensor, the opening sensor is one of an electromechanical switch, a light sensor, a proximity sensor, a capacitive sensor, and an infra-red sensor.

28. The medicament delivery device package assembly as claimed in claim 26, wherein the medicament delivery device package assembly comprises a movable lid arranged on the body; wherein the lid is movable between a lid closed position, in which the opening is covered by the lid; and an lid open position, in which the opening is not covered by the lid; and wherein the opening sensor is configured to detect a contact between the lid and the body or a movement of the lid from the closed position to the open position.

29. The medicament delivery device package assembly as claimed in claim 28, the movable lid comprises a safety lock; wherein the safety lock is at least one of a mechanical lock, an electromagnetic lock, a magnetic lock, and an electrometrical lock.

30. The medicament delivery device package assembly as claimed in claim 16, comprising a temperature sensor, wherein the temperature sensor is configured to detect an ambient temperature and/or a storage temperature within the medicament delivery device package assembly, and/or a temperature of the medicament contained within the medicament delivery device that is stored within the medicament delivery device package assembly; wherein the temperature sensor is always in an activated state when the general switch has been switched on.

31. A system to monitor a medicament delivery device package comprising:
a storage unit with a door, where the door is movable between a closed position such that an inner space of the storage unit is covered by the door and an open position where the inner space of the storage unit is not covered by the door;
a body defining part of the medicament delivery device package and having an interior reservoir for containing a medicament delivery device, where the medicament delivery device package is sized to fit in the inner space;
a motion proximity sensor attached to the body, where the motion sensor detects:
an opening movement of the door from the closed position to the open position;
a change of a distance between the door and the body; and
a presence or an absence of movement of the door in a detectable range of the motion sensor.

32. The system of claim 31, wherein the motion proximity sensor is configured to wake up a set of electronics inside the medicament delivery device package from a sleep mode to a working mode such that a communication unit is activated and sends out a signal.

33. The system of claim 31, wherein the medicament delivery device package comprises a reservoir sensor that is arranged within the interior reservoir such that the reservoir sensor detects a removal of a medicament delivery device from the interior reservoir.

34. The system of claim 33, wherein the medicament delivery device package further comprises an opening, connected to the interior reservoir and an opening sensor that detects when the opening is covered.

35. The system of claim 34, wherein the medicament delivery device package further comprises a temperature sensor that detects an ambient temperature within the interior reservoir, where the temperature sensor is always in an activated state when a set of electronics inside the medicament delivery device package has awoken from a sleep mode.
